(19) United States
(12) Patent Application Publication
(10) Pub. No.: US 2019/0038418 A1
(43) Pub. Date: Feb. 7, 2019

(54) Fastening System

(71) Applicant: SmartScrew Pty Ltd, Kew East (AU)

(72) Inventors: Michael Eliopoulos, Watsonia (AU); Matthew Wildoer, Glen Iris (AU); Narayan Powderly, Balwyn North (AU)

(21) Appl. No.: 16/153,769

(22) Filed: Oct. 7, 2018

Related U.S. Application Data
(63) Continuation of application No. PCT/AU2018/050509, filed on May 25, 2018.

(30) Foreign Application Priority Data
Jun. 1, 2017 (AU) 2017902090
Mar. 9, 2018 (AU) 2018900797

(51) Int. Cl. A61F 2/30; A61C 8/00

(52) U.S. Cl. CPC A61F 2/30749; A61F 2/389; A61C 8/0068; A61F 2/30734

(57) ABSTRACT

A system for fixing a prosthetic component to an anchor implanted in the bone of a patient. The prosthetic component includes a fixing channel and an installation channel. The fixing channel opens to an engagement collar configured to engage with a bone anchor. The installation channel opens from an outer surface of the prosthetic and communicates with the fixing channel. The installation channel permits placement of a fastener therethrough into the fixing channel to secure the prosthetic to the bone anchor. The axes of the fixing channel and the installation channel intersect at a point which lies within the radius of a fastener seat which is configured to engage a head of the fastener when the fastener is placed into the fixing channel.

The present invention relates to a system for fixing a prosthesis to an anchor/implant, and a method of assembling a prosthetic system.

### BACKGROUND

The use of prosthetic implants for surgical implantation in or on a bone of a patient is long established. Thus, in the case of a damaged or diseased hip joint, implantation of a prosthetic femoral implant, a prosthetic acetabular implant, or both, is a common surgical procedure. Another operation frequently performed by orthopaedic surgeons is knee replacement, in which a tibial implant is inserted in the top of a patient's tibia, while one or both condylar surfaces of the patient's femur may be replaced at the same by corresponding femoral implants. In case of accidental fracture of long bones bone plates may be used to stabilise and strengthen the bones. Shoulder, finger, ankle, and elbow replacement prostheses are also known.

Another use of prosthetic implants is in dentistry. A dental prosthesis is a dental device fitted inside the mouth to restore intraoral defects such as damaged, deteriorated or missing teeth or parts thereof. In these situations, dental prosthetic devices, or prostheses, can rehabilitate mastication, improve dental aesthetics and aid in speech recovery.

A dental prosthesis can be made in various forms including crowns (consisting of a single tooth), a bridge (consisting of two, three or more teeth) or a bar (that can provide four or more teeth and even an entire jaw full of teeth). Fixing dental prostheses inside a mouth is usually achieved by two main techniques: connecting the dental prosthesis to adjacent, good teeth or fixing the prosthesis to an implant/anchor that has been fixed into, and interfaces with, the jaw bone. In both techniques the dental prosthesis can be made to be either removable or permanently fixed in the mouth.

In all of these surgical procedures the prosthetic system is intended to bear load. Thus, the objective is to fix the prosthetic system into or onto the bone into or onto which it is implanted as firmly as possible and so as to maintain the bone under loading conditions which are as near as possible to those prevailing the undamaged bone. This is because living bone tissue, which is continually being dissolved and redeposited by the body, tends to disappear unless it is maintained under the appropriate loading level.

While bone cement is frequently used to affix a prosthetic system to the bone, it is generally recognised that, if possible, it is better to avoid the use of bone cement. One benefit of avoiding the use of bone cement is that there is a greater opportunity for the implant to form a bond with the surrounding bone.

In the case of dental prostheses and implants, generally, dental adhesive or screw fasteners are used to permanently fix dental prosthetics to adjoining teeth or to dental implants. The problem with using dental adhesive is that if the prosthesis needs to be removed or replaced the entire prosthesis often needs to be destroyed. Screw fasteners are preferred because they allow for relatively easy removal of the dental prosthesis without destroying the prosthesis itself.

A further variation on prosthesis technology, particularly when fixing to dental implants, is that the prosthesis can be attached either directly or indirectly to the anchor in the bone. An indirect attachment requires an abutment device (colloquially called an ‘abutment’), which is a small interconnecting piece placed between the prosthesis and the anchor. Generally, the abutment is cemented to the prosthesis and then screwed to the anchor thereby fixing the prosthesis to the anchor.

The average lifespan of conventional prosthetic implants is about 15 years. Therefore, if a prosthetic implant is to be implanted into an elderly patient, such a working life span is not problematic as the prosthetic implant might reasonably be expected to function for the remaining lifetime of the patient. However, when such a prosthetic implant is implanted into a young patient, it is likely that one or more revision operations will have to be performed during the lifetime of the patient. It is highly undesirable to subject patients repeatedly to the trauma of such a major surgical procedure as is required to replace a failed prosthetic implant.

The problems sought to be overcome by the prosthetic system described hereafter is that manufacturing such prosthetic devices as those described above is complex and expensive, and at times prone to failure in that unintentional detachment inside the body can occur. Furthermore, consideration must be given to the medical practitioner fitting the prosthetic system in a patient's body, and to make the fitting process as straight forward as possible to minimise incorrect placement, and strain on both the practitioner and patient.

### SUMMARY OF INVENTION

In accordance with the present invention there is provided a system for fixing a prosthetic component to an anchor implanted in bone of a patient, the system comprising a prosthetic component having a fixing channel and an installation channel; and a fastener that, in use, is located in the prosthetic component for fastening the prosthetic component to the anchor. The fastener has a spherical or part spherical head. The fixing channel opens at one end to an anchor interface side of the component and an opposite end opens internally of the component to meet the installation channel at a fastener seat. The fixing channel is configured to accommodate there-through a shaft of the fastener, while the head of the fastener sits on the fastener seat. The fastener seat is profiled to receive, and have substantially the same radius of curvature, as the fastener head. The installation channel opens to an upper side of the prosthetic component and has an installation axis that is inclined relative to a fixing axis of the fixing channel, where the axis of the fixing channel intersects the installation axis at a point that lies within the radius of the fastener seat. The fixing channel at the anchor interface side can define a collar of the prosthetic component configured to be inserted below a bone line into the anchor.

The advantage of providing a spherical-shaped fastener seat having a radius of curvature that is substantially the same as the fastener head is that when the fastener, which may be a screw, is held under tension fastening together the prosthetic component to the anchor, the tolerance between the fastener head and seat is tight and effectively locks the fastener against twisting loose. In particular, the normal contact force between the underside of the fastener head and the spherical seat increases as the contact point moves from a point adjacent the fixing channel to a point at the widest part of the semi-spherical head. At this widest point the friction forces against the fastener loosening are at their largest. A tight engagement between the fastener head and seat can be further attained by pressure, or compression, between the contacting surfaces as a result of the material of the fastener and prosthetic component having slight elasticity and/or resilience.

Furthermore, having the intersection of the fixing channel axis and the installation channel axis within the radius of the fastener seat provides for direct access of rigid machining tools to more accurately and directly machine the fastener seat to the correct geometry, namely to be substantially the same radius of curvature as the head of the screw fastener head. In some embodiments, the seat could be machined with the same tool, and at the same time, as the installation channel is created. Furthermore, installation of the screw onto the fastener seat can be performed with relative ease as a straight screwdriver can directly access the screw sitting on the seat.

The installation channel will preferably have a diameter that is at least equal to twice the radius of curvature r of the fastener seat.

In one embodiment, the fastener seat has an initial hemi-spherical geometry such that tangent lines extending normally to the diameter of the hemisphere of the hemi-spherical geometry will be parallel to the installation axis. Accordingly, the tangent lines of the hemi-spherical seat define a seat orientation axis that is co-linear with the installation axis.

It is understood that while during manufacture of the prosthesis the fastener seat may be initially machined to have an initial hemi-spherical geometry with the above-described parameters, the seat may subsequently be machined as part of widening the installation channel to have a semi-spherical geometry that has a circumference that is less than hemi-spherical.

Preferably, the installation axis is inclined relative to the fixing axis at an angle θ of between more than 0° and 50°, and preferably 0°≤θ≤35°, and 15°≤θ≤25°, and in one embodiment is approximately 20°. The angle of inclination will, of course, depend on the shape of the prosthetic component, and on the ease of accessibility whether the access/entry point of the installation channel is on the distal side or the proximal side, which will dictate ease of access for the installer (orthopaedic surgeon, dentist, etc), and/or an entry point that is hidden as much as possible from view. This becomes more relevant when the prosthetic component is visible after being secured to the anchor, such as in the case of a prosthetic tooth.

In one embodiment, as indicated above, the prosthetic component may be a prosthetic tooth. In this aspect, the anchor is configured for placement in a jaw bone. The prosthetic component in this aspect is preferably shaped and sized to resemble a mammalian tooth and can be made to resemble the form of any tooth or variation thereof found in a mammalian mouth. The prosthetic component may alternatively be configured as a single piece to represent a row of two or more adjacent teeth and could even be made to resemble an entire row of teeth.

In one embodiment, the prosthetic component is a prosthetic joint or at least a portion of a joint, and the anchor for use in this embodiment may be configured for placement in a femoral head. In this embodiment, the system of the invention is used in hip replacement surgery, in the case of a damaged or diseased hip joint, implantation of a prosthetic femoral implant, a prosthetic acetabular implant, or both.

In another embodiment, the prosthetic component may be a prosthetic joint or at least a portion of a joint, and the anchor for use in this embodiment may be configured for placement in phalangeal bone. In this embodiment, the system of the invention may be used in hand surgery to replace diseased or damaged knuckles.

In another embodiment, the prosthetic component may further include at least a second fixing channel and at least a second corresponding installation channel, and the system further includes at least a second fastener. In this embodiment, the system of the invention may be used in knee replacement surgery, in which a tibial anchor is inserted in the top of a patient's tibia, while one or both condylar surfaces of the patient's femur may be replaced at the same time by corresponding femoral anchors.

In an aspect of the invention, the installation channel of the system of the invention may have a maximum width, the fastener having a maximum width greater than 90% of the maximum width of the installation channel.

In another aspect of the invention, the head of the fastener may have a depth along a central longitudinal axis of the fastener, the fastener including a tool-engaging opening having a depth at least as great as the depth of the head. The head may also have a radius of curvature less than 90 degrees relative to a central longitudinal axis of the fastener.

The head of the fastener may include a shaft having a root diameter which decreases and increases along a central longitudinal axis of the fastener, and may be configured to directly contact an internal surface of the prosthetic component to clamp the prosthetic component to the anchor.

In one aspect, the installation channel may include a depth along the installation axis, the fastener including a shaft having a length greater than the depth of the installation channel.

In another aspect, the said prosthetic component includes a fastener seat having a maximum diameter less than a maximum diameter of the head of the fastener.

The fastener of the system of the invention may include a shaft having a length, the fixing channel of the prosthetic component having a length along the fixing axis that is longer than a majority of the length of the shaft of the fastener.

In one aspect the prosthetic system comprises the fastener, which is preferably a screw fastener having a screw head atop a shaft where an underside of the screw head is semi-spherical in profile to have a radius of curvature that substantially matches that of the fastener seat.

In another embodiment the prosthetic system is indirectly fixed to the implant through an intermediary member, namely an abutment. The abutment in this embodiment forms the lower part of the prosthetic component.

In accordance with the present invention there is also provided a method of fixing a prosthetic component to an anchor in a patient. The method includes machining a fixing channel into an anchor interface side of a prosthetic component, wherein the fixing channel at the anchor interface side may define a collar of the prosthetic component configured to be inserted below a bone line into the anchor, and wherein the fixing channel terminates internally of the component. The method also includes machining an installation channel from an upper side of the prosthetic component that meets the fixing channel internally of the component. Also included is machining a fastener seat through the installation channel at the juncture of the fixing channel and installation channel, including profiling the fastener seat to have a radius of curvature that is substantially the same as a spherical or part spherical head of a fastener adapted to be accommodated on the fastener seat through the fixing channel, where the fixing channel has a fixing axis that intersects an installation axis of the installation channel at a point that lies within the radius of curvature of the fastener seat. The collar of the prosthetic component, when present, is inserted into the anchor, alternatively the anchor interface side of the prosthetic component is inserted into the anchor; and a fastener is inserted through the installation channel into the fixing channel to engage the fastener head with the fastener seat and fix the prosthetic component to the anchor.

In one embodiment, a cutting tool is first used to create an installation hole along the installation axis defining the entry point in the prosthetic component, the orientation and angle of inclination of the installation channel. That hole is subsequently widened by another tool, such as an endmill, twist drill or reamer, to form the installation channel.

In an alternative embodiment, the installation channel is formed in a single operation in the solid prosthetic component. Such a tool that could be used in this embodiment would be an endmill. One example of a suitable endmill is a ball nose endmill, although a bull nose endmill and a lollipop endmill could also be used, as could any other tool having a substantially spherical end.

In a further embodiment, the installation channel may be cut in a manner that widens the channel laterally of the installation axis to create a cut section above the fastener seat that can be used to give the cutting tool greater space to machine the fastener seat that has a seat orientation axis that is co-linear with the fixing axis. This is different to the embodiment described further above where the seat orientation axis is co-linear with the installation axis.

The same cutting tool described above can be used to machine the fastener seat with the tool extending through the installation channel. Alternatively, another finishing tool could be used to machine the seat as a subsequent step to the initial step of machining the installation channel.

In the above embodiments, the cutting tool is preferably a straight, rigid machining tool, and could be a twist drill or a milling tool.

The method of making the prosthetic system can be made by hand by a skilled technician, or could be made automated through use of computer-aided manufacturing (CAM) in a CNC milling machine. Such automated manufacturing can be associated with a controller as part of a system with the use of CAD (computer aided design) software.

According to another aspect of the invention there is provided a system of making a prosthetic component for fixing to an anchor, comprising inputting into a controller, prosthetic operating parameters, wherein the controller generates design data on the geometry of the prosthesis component and calculates tool cutting sequences; and transmits the generated design data to a machining centre to machine a prosthetic component according to the prosthetic system described above.

In an embodiment the prosthetic operating parameters can include location and orientation of fixing axis relative to prosthetic component, location and orientation of installation axis relative to prosthetic component, anchor interface geometry of the prosthetic component, point of entry of installation channel, radius of curvature of fastener seat. The prosthetic operating parameters may be in the form of a scanned model.

The design data could include fixing channel geometry, installation channel geometry and fastener seat geometry. The tool cutting sequences could include the calculation of machining operations including machining paths, machining cycles and interpolated milling operations.

Further embodiments of the system of the invention may include use of the system in non-biological applications, for fixing mechanical components. The system of the invention allows for removal of flanges of material used for bolted connections between components, resulting in reduced material usage, and an increase in part strength. The system of the invention may, for example, be used in the oil and gas industry for pipelines and drilling equipment; in pressure vessels; in shafts, wind turbine blades; and in the aerospace industry for pipe joints and mechanical connections on wings and control surfaces.

### BRIEF DESCRIPTION OF THE DRAWINGS

Embodiments, incorporating all aspects of the invention, will now be described by way of example only with reference to the accompanying drawings in which:

FIG. 1(a) is a front view of a dental prosthetic system attached to an anchor in accordance with an embodiment of the present invention;

FIG. 1(b) is a side sectional view of the dental prosthetic system taken at section 1-1 of FIG. 1(a);

FIG. 2(a) is a front view of a dental prosthetic system attached to an anchor in accordance with another embodiment of the present invention;

FIG. 2(b) is a side sectional view of the dental prosthetic system taken at section 2-2 of FIG. 2(a);

FIG. 3(a) is a front view of a dental prosthetic system attached to an anchor in accordance with a further embodiment of the present invention;

FIG. 3(b) is a side sectional view of the dental prosthetic system taken at section 3-3 of FIG. 3(a);

FIG. 4(a) is a front view of a dental prosthetic system attached to an anchor in accordance with a still further embodiment of the present invention;

FIG. 4(b) is a side sectional view of the dental prosthetic system taken at section 4-4 of FIG. 4(a);

FIG. 5 is a side sectional view of application of an embodiment of the invention to a knee replacement;

FIGS. 6(a), 6(b) and 6(c) respectively show side views of a bull nose endmill, a ball nose endmill and a lollipop tool;

FIGS. 7(a) and 7(b) are respectively isometric and side views illustrating an example of a fastener of the prosthetic system; and

FIG. 8 illustrates a schematic block diagram showing the process of making a prosthetic system in accordance with the invention.

### DETAILED DESCRIPTION

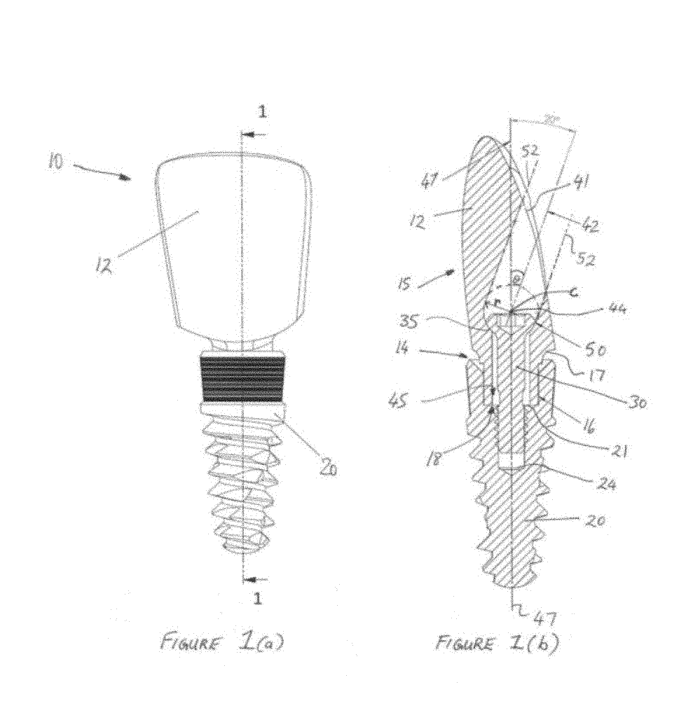

A prosthetic system is illustrated in FIGS. 1(a) to 4(b) and is generally denoted by the reference numeral 10. The prosthetic system 10 described herein is adapted to be fixed to an anchor 20 that is itself fixed into bone of a patient. Referring to FIGS. 1(a) and (b), exemplifying a dental prosthetic system, the prosthetic system 10 comprises a prosthetic component 12 having an upper part 15 that is the part of the component that will be exposed above a patient's bone line once attached to the anchor 20. At a lower part 14, the prosthetic component 12 narrows down to a collar 16 that is adapted to locate in an interfacing recess 21 of the anchor 20. The collar 16 and anchor recess 21 are complementarily profiled to mesh one inside the other. Specifically, a shoulder 17 transitions from the upper part 15 of the prosthetic component 12 to collar 16 at the lower part 14 of the prosthetic component, which lower part defines an anchor interface side 18 which, in use, is hidden from view at or below a bone line.

In other embodiments, the component 12 does not include a collar 16, and the anchor interface side 18 is complementary to the interfacing recess 21 of the anchor 20.

The prosthetic system includes the prosthetic component 12 and a fastener 30 where the fastener is adapted to be located inside the prosthetic component for fastening the prosthetic component 12 to the anchor 20. The prosthetic component furthermore comprises two channels. An installation channel 40 is provided through which fastener 30 is inserted in order to correctly locate the fastener inside the prosthetic component. A fixing channel 45 is also provided to support a shaft 32 of the fastener 30 to extends through the fixing channel 45 to outside of the anchor interface side 18 and into a threaded fastener receiving hole 24 into which the fastener is tightened to fasten the prosthetic system 10 to the anchor 20.

With the fixing channel 45 opening at one end of the channel to the lower end of the prosthetic component at the anchor interface side 18, an opposite end of the fixing channel opens internally of the prosthetic component 12 to meet the installation channel 40 at a meeting point, which is a fastener seat 50. The installation channel 40 with one end terminating internally of the prosthetic component at fastener seat 50, opens at its other end on a distal side of the upper part 15 of the prosthetic component 12.

In the instance where the system of the invention is used for dental prosthetics, the installation channel 40 opens rearwardly of a generally vertical axis of the prosthetic system, where a forwardly direction faces outwardly of a patient's mouth. An entry point 41 of the installation channel where it opens on the prosthetic component is aimed to locate rearwardly and toward the distal side of the patient's mouth so that the entry 41, even when plugged into the surface of the prosthetic component after installation, is less visible, if at all, by a person looking at the patient's teeth.

With the fastener seat 50 located roughly at a lower centre of the prosthetic component 12, the installation channel has an installation axis 42 that is likely to be inclined relative to a fixing axis 47 of the fixing channel 45. The fixing axis is the axis along which the fixing channel extends and on which the shaft 32 of the fastener 30 lies. The fixing access 47 generally defines a vertical axis of the prosthetic system 10.

The fastener seat 50 has a profile that is adapted to receive a spherical or part spherical head 34 of the fastener 30 which sits on and is tensioned against the fastener seat 50. To provide an even distribution of tension forces between the fastener head 34 and the fastener seat 50 the radius of curvature of the fastener seat is substantially the same as the radius of curvature of the underside 35 of the fastener head 34.

In the embodiments illustrated in the figures, the fastener is a screw and will be referred to hereinafter as a screw or fastener. The underside 35 of the screw head 34 is spherical in nature while the screw head itself may be part spherical or a full sphere or a body resembling a part or full sphere, such as a part or full ellipse. Regardless, the spherical or rounded, underside of the screw head 34 will sit on fastener seat 50, which has a matching profile to the underside 35 of the screw head and can be properly tensioned against the fastener seat 50 with substantial avoidance of point stresses in order to more easily insert and then retain the prosthesis 10 on the anchor 20.

The underside 35 of the screw head 34 at the top of screw shaft 32 is part or semi-spherical in profile as described above, and curves convexly from the shaft 32 with a radius of curvature that is substantially the same as the fastener seat. The screw head underside 35 curves toward an inwardly facing chamfered circumferential edge 36 that meets a flat top surface 38. The flat top surface 38 is shorter in length than the maximum width/diameter of the screw head. A drive slot 39 is provided in the top surface 38 to receive a screw driver. The drive slot 39 could be any type of drive slot used in dental hardware, such as a hex drive slot. The semi-spherical underside 35 of the head with the chamfered edge 36 provides a robust screw that can withstand the tension of being fixed into the anchor, and that is also profiled to be readily installed through the installation channel onto the fastener seat.

As illustrated in the figures, the prosthetic system 10 is configured so that the fixing axis 47 intercepts the installation axis 42 at a point of intersection 44 that lies within the radius of curvature r of the fastener seat. Such a configuration where the installation and fixing axes intercept within the radius of curvature of the fastener seat, which is substantially similar to the radius of curvature of the fastener head 34 and more specifically to the underside 35 of the fastener head, provides for a stronger and more reliable prosthetic system 10 that is less prone to failure once installed in a patient's bone.

During prosthetic insertion by a medical professional, the configuration provides for ease of insertion by an operator of the driver tool through the installation channel to properly locate on the fastener seat 50. The installation channel provides direct access for the screw and the screw driver for relatively quick and simpler attachment of the prosthetic system to the anchor in a patient. Furthermore, during manufacture, the configuration of the channels 40, 45 allow for direct access to rigid machining tools that are able to directly machine the fastener seat to the correct geometry for receiving a matching rounded screw 30. On this point, there is an advantage in that the installation channel and the fastener seat can be, if desired machined with the same tool in the same operation.

As can be particularly seen in FIGS. 1(b), 2(b), 3(b) and 4(b) the installation channel has a diameter that is at least equal to twice the radius of curvature r of the fastener seat.

As the screw head 34, or at least the underside 35 thereof, has a radius of curvature that matches and meshes with the radius of curvature of the fastener seat 50, the above equation represents the clearance access in the installation channel 40 required for a screw 30 to pass through to gain access to the fastener seat 50.

The radius of curvature “r” of the fastener seat (and therefore also of the screw head 34) is shown in the drawings and the area bound therein defines the intersection point between the fixing axis 47 and installation axis 42. The axes 42, 47 may intercept anywhere within the sphere defined by the radius of curvature of the fastened seat when viewed and calculated from any perspective. While benefits of the presently described prosthetic system 10 can be derived with the intersection 44 of the axes falling anywhere within a three dimensional radius of curvature of the fastener seat 50, it is expected that the better results in terms of accuracy of manufacture and ease of installation are achieved when the fixing and installation axes 42, 47 intersect at an intersection point 44 that lies close to or at the centre, c, of the radius of curvature.

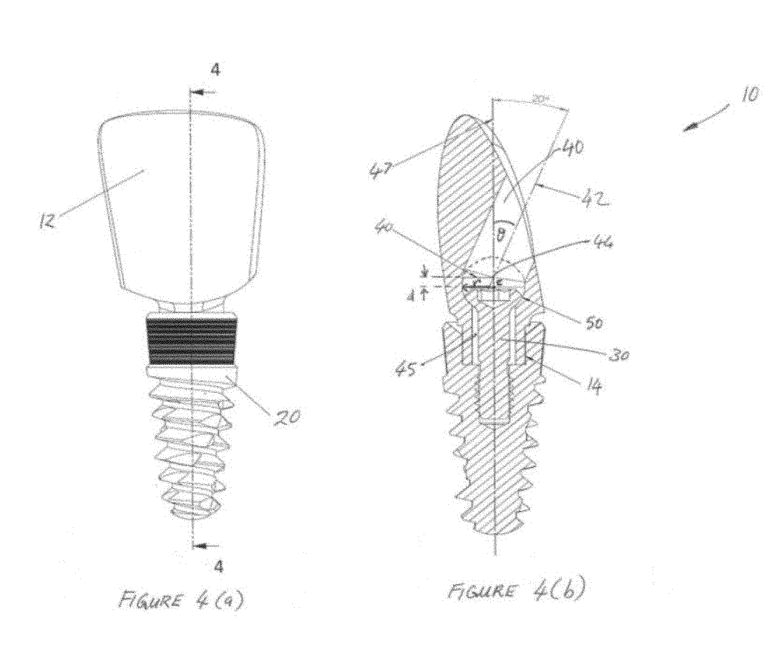

FIGS. 1(b), 2(b) and 3(b) illustrate intersection point 44 illustrated as lying close to the centre point of the radius of curvature r of the fastener seat 50. In FIG. 4(b) the intersection point 44 lies offset and a little higher from the centre point c because the fastener seat 50 is machined to be a little “dropped” relative to the internal end point of the installation channel 40, which in FIG. 4(b) is at intersection point 44. A drop in seat 50 could be machined using a lollipop tool (discussed in more detail below) which has an end that is larger than its shaft and consequently is able to be angled in the installation channel to machine the dropped fastener seat.

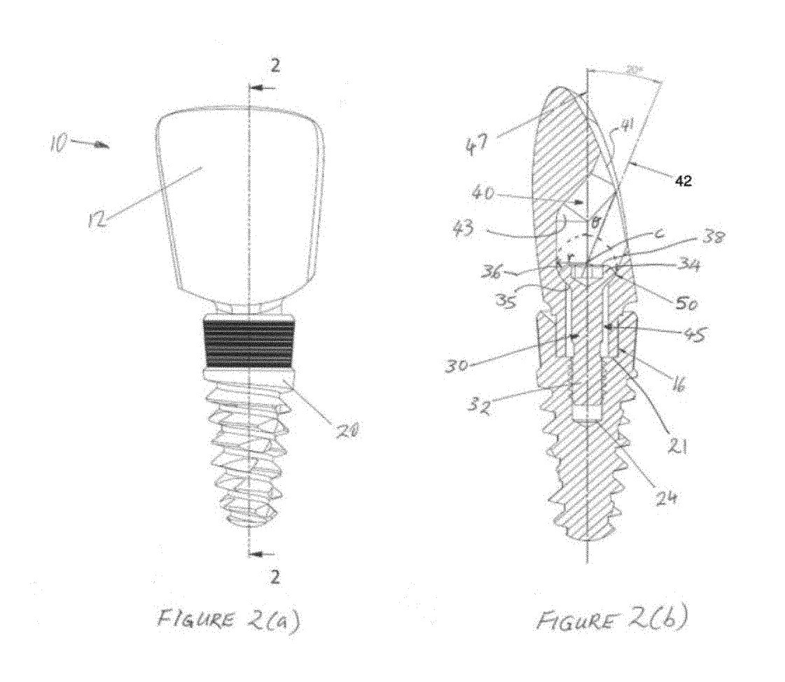

The embodiment illustrated in FIGS. 1(b) and 2(b) have the fastener seat 50 initially oriented along the direction of the installation axis 42. In order words taking the hemi-spherical geometry of the fastener seat 50 in FIG. 1(b) and extending tangent lines 52 in a direction normal to the diameter of the hemisphere of the fastener seat geometry will produce parallel tangent lines 52 that are parallel with the installation axis 42. In FIG. 1(b) the parallel tangent lines 52 also define the side walls of the installation channel 40. Accordingly, the fastener seat 50 in the embodiment illustrated in FIGS. 1(a) and 1(b) is oriented along the direction of the installation 42.

The hemispherical geometry of the fastener seat described above relates to an initial hemispherical geometry. That is, a geometry that is initially machined in order to define the radius of curvature r of the fastener seat, even if it is subsequently machined. This initial geometry comes from an understanding that after initially machining the fastener seat may subsequently be widened as part of a widening of the installation channel 40, for example as illustrated FIG. 2(b) so that the initial hemispherical geometry of fastener seat is made into a semi spherical geometry (where the curved underside 35 covers a circumference of less than 180°). At that point the tangent lines 52 will no longer be parallel but it is understood that a fastener seat orientation can still be calculated by measuring tangent lines taken from the initial hemispherical sit geometry.

Again, FIG. 2(b) illustrates a fastener seat 50 having an orientation along the installation axis 42 but also having a cut out 43 that has been machined to widen the installation channel 40 to provide greater clearance to machine fastener seat 50, and inherently provides greater room to insert and position screw 30 during attachment of the prosthetic system 10 to an anchor 20.

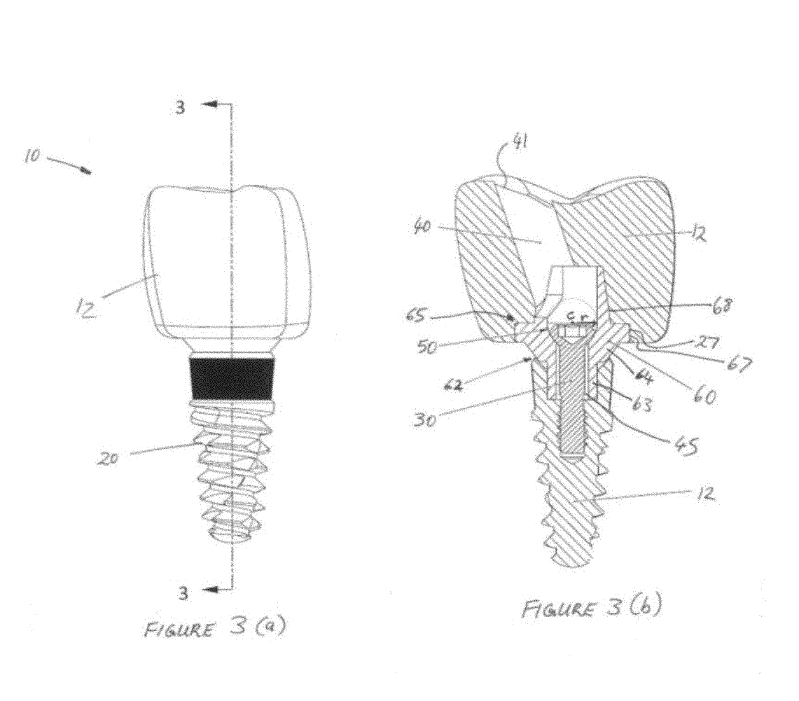

Conversely, the fastener seats 50 in the embodiments of FIGS. 3(b) and 4(b) are oriented along the fixing axis 47 and therefore align along the vertical axis of the prosthetic component 12 (which is also the fixing axis 47 in these cases). FIG. 4(b) illustrates an embodiment where the fastener seat had a seat orientation axis that initially aligned with the installation axis 42 but then was further machined to drop the fastener seat 50 by depth of d. The further machining reoriented the seat orientation axis along the vertical fixing axis 47. The dropped seat of this embodiment has the advantage of providing a greater head clearance for the screw and is suitable for use when using a short screw.

In the embodiment of FIG. 3(b) the fastener seat is provided with an intermediate member, and namely an abutment 60. The abutment 60 forms the lower part 14 of the prosthetic component 12 and allows for greater freedom of movement and slight deformation during installation in positioning and screwing the prosthesis on the anchor. This is achieved because the abutment is cemented to the upper part 15 of the prosthetic component which allows for material flexibility of the prosthetic component that would not necessarily be achievable with, for example, a ceramic prosthetic.

The abutment comprises an anchor interfacing side 62 with the anchor 20 that is profiled with a collar 63 and shoulder 64 to complementarily locate in the anchor's interfacing recess 21, similar to that described above in relation to the direct contact embodiment where the prosthetic component 12 is formed as a single piece and is directly attached to the anchor 20 without any intermediary member. On an opposite side the abutment has a body interfacing profile 65 that also has a shoulder 67 and collar 68 to locate within a complementarily profiled abutment recess 27 in the prosthetic component, where that abutment recess opens into the installation channel 40.

The abutment 60 is cemented to the upper part of the prosthetic component, which forms the visible part of the prosthesis in use, and is not generally visible once installed in a patient's body. In this embodiment, the abutment would form the lower part 14 of the prosthetic component as strictly defined, so that the upper part 15 and abutment 60 as shown in FIGS. 3a and 3b form the ‘prosthetic component’. However, for ease of understanding in describing this embodiment, and for consistency with general understanding in the dental industry, the upper part 15 of the prosthetic component illustrated in FIGS. 3a and 3b is referred to as the ‘prosthetic component 12’ attached to the abutment 60.

In the embodiment of FIGS. 3a and 3b, being an example where the prosthetic system of the invention is in the shape of a mammalian molar tooth, the abutment 60 comprises the fastener seat 50 on which the screw 30 is supported for fixing to the anchor. The fixing channel 45 extends entirely through the abutment and accommodates the fastener seat 50 that opens into the installation channel 40 through a cut away 69 to one side of the abutment 60. In practice, the fastener seat 50 would likely be machined in abutment 60 before the abutment is fixed to the upper part of the prosthetic component, but it could be machined after fixing the abutment and the prosthetic component.

The fixing axis 47 and the installation axis 42 are inclined relative to each other, where the fixing axis 47 is shown in the accompanying drawings as the general vertical axis of the prosthetic system 10. The axis of inclination of the installation axis relative to the fixing axis is shown as angle θ. Angle θ will necessarily vary depending on the shape and structural geometry of the prosthetic component to be implanted. The shape will obviously vary depending on the size and shape of the prosthetic component.

For example, FIGS. 1(a) and 1(b) show a prosthetic component 12 long and slender in shape to resemble a front upper or front lower human tooth. The angle of inclination θ in this embodiment is approximately 20°.

In contrast FIG. 3(a) and FIG. 3(b) illustrate the prosthetic component as a molar tooth which requires a squarer component with a broader base than the longer component of the front tooth in FIG. 1(a) and FIG. 1(b). In FIG. 3(b) the entry point 41 of the installation channel 40 is offset from the vertical fixing axis 47 to, in use, be hidden from view as much as possible in a downward face towards a centre crevice in the upper surface of the tooth. Accordingly, the installation channel with its installation axis 42 extending between the entry 41 and fastener seat 50 will have an inclination that will depend upon the location of entry 41. In FIG. 3(b) the inclination angle θ is approximately 20°.

In practice angle θ can be more than 0° and is likely to be less than 50°. In a practical embodiment θ is between 0° and 35°, and for a majority of cases will be between 15° and 25°, and at approximately 20°. It is however understood that the angle of θ will vary as discussed above dependent on the shape and geometry of the tooth that the prosthetic component is cut to resemble.

it will be appreciated that aspects of the inventive concept may be configured for other areas and fields. For example only, in paediatrics, and more particularly, in joint replacement, such as hip, knee and knuckle replacement, the aspects of the inventive concept may be adapted to reduce wear in a replacement joint.

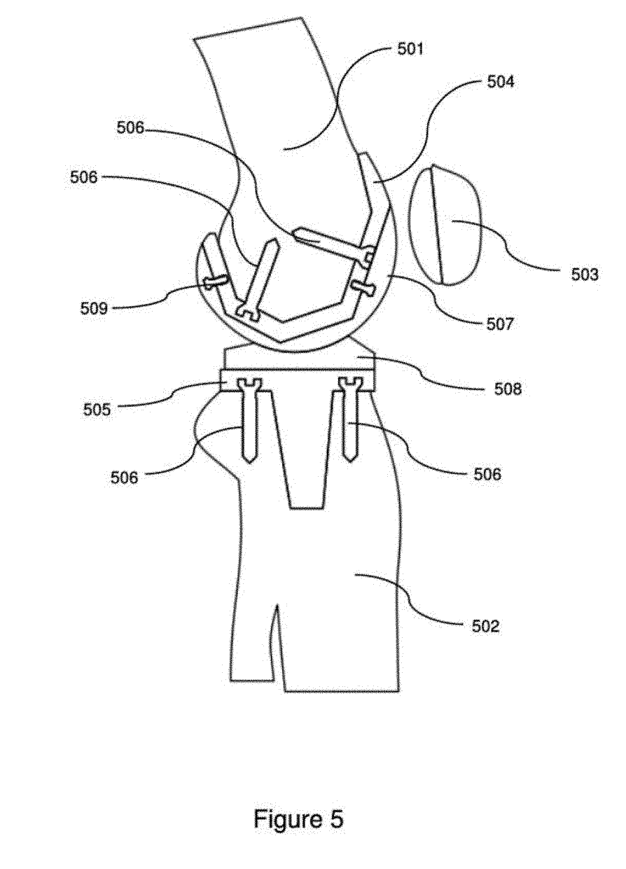

The embodiment of the invention illustrated in FIG. 5 is directed to the use of the invention in a knee replacement embodiment. FIG. 5 is a cross sectional side view of a knee joint, including head of femur 501, head of tibia 502, and patella 503. In the embodiment, a femoral sub-plate 504 and tibial sub-plate 505 are attached to the respective femur 501 and tibia 502 using conventional bone screws 506. The wear surface 507 and tibial wear surface 508 are then attached to the respective sub-plates using fasteners 509 according to the invention. By using the system of the invention in this application, the insertion channel and fasteners are directed away from the wear surface, minimising possible effects on those wear surfaces.

The materials of the components of the system 10 may vary according to cost, function, aesthetics, workability, availability and other factors. For example, in a prosthetic system for dental application, the prosthetic component can be made from any material with properties appropriate for dental requirements, such as resin (acrylic or other), porcelain, appropriate plastics, composites, metals including gold, titanium and alloys, and carbon fibre.

Similarly, the fastener 30 of the invention can be made from any material with properties appropriate for dental requirements, such as resin (acrylic or other), porcelain, appropriate plastics, composites, metals including stainless steel, gold, titanium and alloys, and carbon fibre. The fastener can of the same as, or different material to the prosthetic component.

In embodiments of the invention used in other applications, such as hip, knee or other joint implant surgery, the materials of the prosthetic component and fastener may be adjusted accordingly.

With the above described prosthetic system 10, the method of fixing a prosthetic component 12 to an anchor 20 can be made more efficient and systemised through the use of standard tools or tool bits. In its basic form, the method includes the steps of:
Machining the fixing channel 45 into an externally machined and prepared solid prosthetic component 12 that resembles or simulates the required prosthesis, for example one or more teeth, or a joint component. The fixing channel 45 is machined from the anchor interface side 18 of the prosthetic component 12 and is machined sufficiently far into the component to terminate at a location that forms the location of the fastener seat 50. The fixing channel 45 at the anchor interface side 14 defines a collar 16 of the prosthetic component configured to be inserted below a bone line into the anchor 20. Any type of known tool for forming a bore can be used to machine the fixing channel. The fixing channel includes a fixing axis that can be used to define a vertical reference axis for the prosthetic system 10.
Machining an installation channel 40 from an upper side of the prosthetic component straight down into the component to meet the fixing channel 45 internally of the component. The installation channel is likely to be inclined depending on where the entry point of the installation channel 40 is selected on the prosthetic component, and the installation channel has an installation axis 42. The entry point may be on the distal or proximal side of the component, or any other preferred location that will provide optimal access for person installing the prosthesis on an anchor. The installation channel should open up to meet the end of the fixing channel internally of the prosthetic component.
The fastener seat 50 is then machined by inserting a tool through the installation channel where the tool is designed to machine a radius of curvature of the fastener seat where the radius of curvature is predefined and is substantially the same as a spherical or part spherical head of a fastener 30 that is adapted to sit on the fastener seat. Furthermore, the channel configuration is such that the fixing axis 47 intercepts the installation axis 42 at a point 44 that lies within the radius of curvature of the fastener seat.
The collar 16, or anchor interface side, of the prosthetic component 10 is then inserted into a corresponding recess in the anchor 20.
The fastener 30 is inserted through the installation channel 40, into the fixing channel 45 to engage the fastener head 34 with the fastener seat 50. The shaft 32 of the fastener 30 to extends through the fixing channel 45 to outside of the anchor interface side 18 and into a threaded fastener receiving hole 24 into which the fastener is tightened to fasten the prosthetic system 10 to the anchor 20.

To reduce changes in tools used and to make the process more efficient, the same cutting tool can be used to machine the installation channel as well as machine the fastener seat. Such a cutting tool that could be used for this single operation could be a specific cutting tool having an end that is capable of forming the semi-spherical seat, and more particularly an end that is substantially spherical in its cutting profile. Examples of such tools include a twist drill having a spherical cap end or a mill having a spherical-shaped end, such as a ball nose endmill.

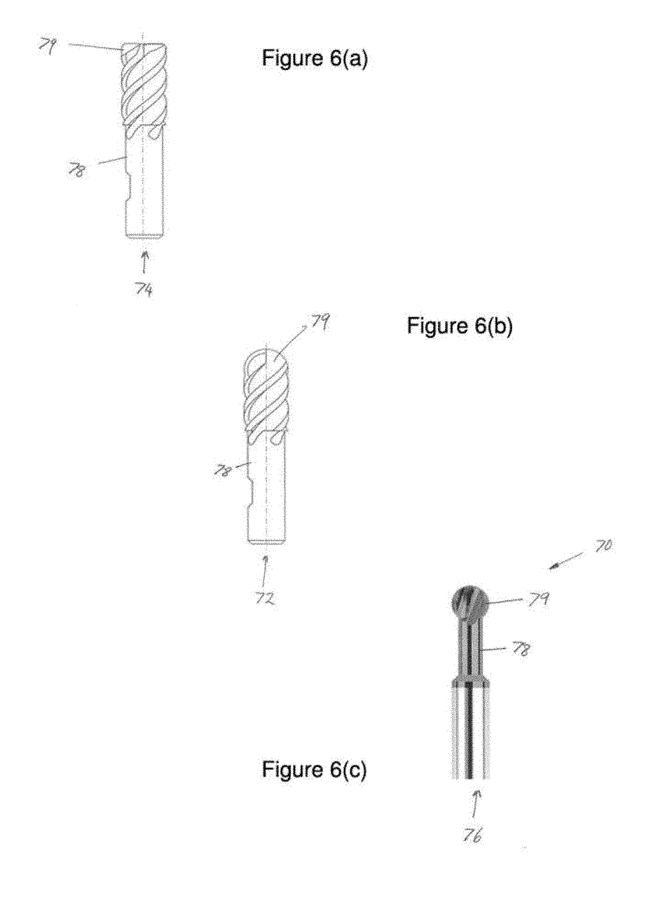

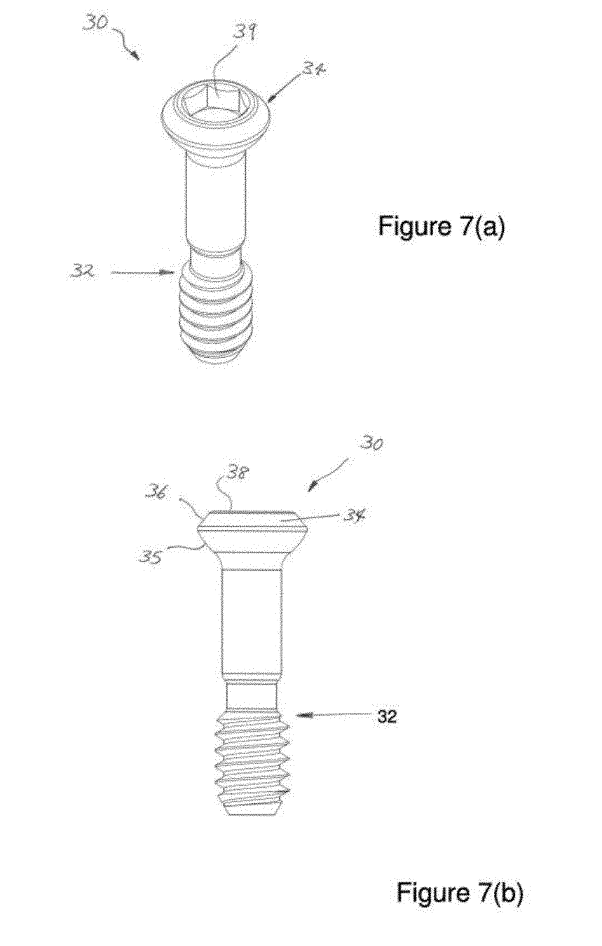

FIGS. 6(a), 6(b) and 6(c) illustrate respectively examples of bull nose, a ball nose, and a lollipop endmill tool. The tools are made of a hard-cutting material, such as solid carbide.

In an alternative embodiment of that described above the installation channel can be made in two or more steps by first machining the channel with a simple cutting tool to create an installation hole along the installation axis. That hole is subsequently widened by another tool to form the installation channel, where that other tool could be a tool as described above that is also used to machine the fastener seat. Alternatively, a third tool could be used to machine the fastener seat.

The tool that is used to machine the fastener seat, and that can also be used to widen or even initially create the installation channel, is a tool with an end profile that can be used to accurately machine a curved surface having a finely precise radius of curvature r, as predetermined to match the same radius of curvature on the underside 35 of a screw head 34 of a screw 30 that will extend through the fixing channel 45.

The end profile of the tool, which will be a twist drill tool, will at least have an element of curvature that can be shown as the illustrated bull nose tool of FIG. 6(a), or a more pronounced curvature as illustrated with the ball nose tool in FIG. 6(b) and the lollipop tool in FIG. 6(c). Such tools can be manipulated through a narrow access of the installation channel to directly reach the fastener seat 50 and to machine the required part-spherical surface of the seat around the internal opening of the fixing channel 45.

The cutting tool used, such as those illustrated in FIGS. 6(a), 6(b) and 6(c) is a straight and rigid tool that has a shank, or shaft, 78 that at least for a section adjacent the end cutting head 79 has a narrower diameter so as to not interfere with the machining process in creating the installation channel and/or fastener seat. The spherically rounded cutting head 79 of the cutting tool 70 extends proud from the shaft 78 and so the rounded end has a larger width/diameter than the adjacent shaft. The shaft may be in the form a collar that can widen into a main shaft part of the tool shaft. The shaft adjacent the head will have a diameter that is the same as or smaller than the diameter or the width (at its widest part) of the rounded cutting head 39. The relative dimension between the cutting head and the shaft allows the cutting tool to be used to both create the installation channel and to machine to a precise finish the fastener seat at the required seat geometry.

As discussed above, the tool used to machine the fastener seat has a spherically-rounded cutting end or cutting head 79. The round profile of the cutting end is used to form the predetermined radius of curvature of the fastener seat. The radius of curvature is predetermined to be substantially the same as the radius of curvature of the underside of a fastener head to be used to fasten together the prosthesis and the anchor. The term ‘substantially’ the same is used herein because while it is desired to attain the same radius of curvature for both the fastener head and fastener seat, it is understood that margin of errors can occur. However, it is desired that the radii of curvature are the same to within 5% or less, and preferably 2% or less.

By way of examples of tools used to machine the fastener seat and, optionally widen or create the installation channel, reference is again made to FIGS. 6(a), 6(b) and 6(c). In general, a lollipop endmill 76 has a rounded head 79 with a cutting diameter that is larger than the diameter of its shaft 78. Ball-nose endmills 72 have shafts 78 that have a diameter that is substantially equal to the cutting diameter of their rounded heads 79. Bull-nose endmills 74 have a flatter cutting head 79 than ball-nose endmills with rounded corners. The cutting diameter of a bull-nose endmill is greater than twice the radius of the rounded corners of the cutting end.

The cutting tools are made of a material suitable to cut the prosthetic component including materials such as chrome cobalt, titanium alloy, acrylics and zirconium.

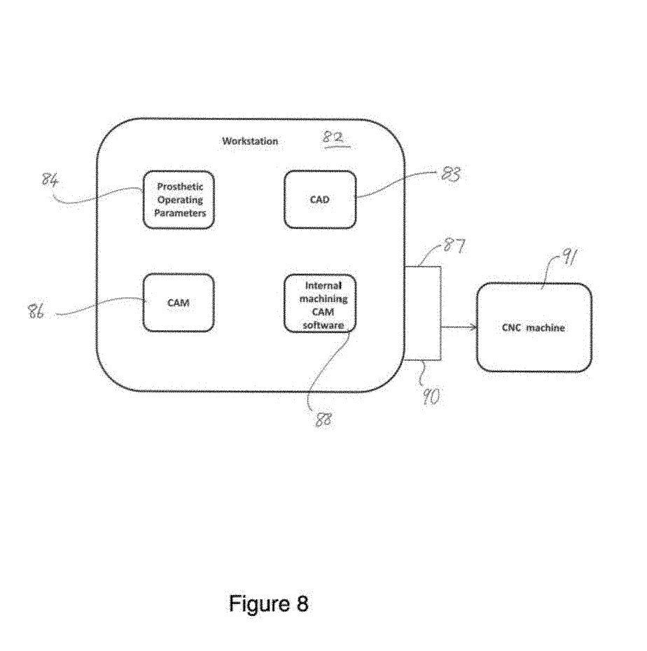

FIG. 8 illustrates in block diagram of the system 80 involved in making the prosthetic system 10 for fixing to an anchor 20. The system 80 includes a controller 82 having a user interface in the form of a scanner, touch screen, keyboard or the like, into which a user can input prosthetic operating parameters 84.

Examples of prosthetic operating parameters which can be provided to the controller 82 include, in the instance of a dental prosthetic system, scanned three-dimensional models of a patient's tooth or teeth structure. The controller may be in the form of a workstation and includes a three-dimensional CAD (Computer Aided Design) software 83 that will generate a prosthetic component design to be cut and machined. Other parameters that are provided into the controller include the interface geometry of the anchor to which the prosthesis is to be attached, which is part of the exterior geometric parameters of the prosthetic component. The controller further allows a user to select the location on the surface of the model of the prosthetic component for the entry point 41 of installation channel 40. The user may also use additional tools in the CAD software to modify the model of the prosthetic component manually, as desired.

The workstation 82 will then calculate and generate design data relating to the external geometry of the prosthetic component. The designed data calculated will include relative location of the entry points for the installation axis 42 and fixing axis 47, and the anchor interface profile 18 that complements the anchor to which the prosthesis will be attached.

Once the design data has been calculated it is exported to a CAM (Computer Aided Manufacturing) 86 program to generate external machining data 87 for the CNC (Computer Numerical Control) machine 91. The CAM 86 generated external machining data will include tool cutting sequences to create the external geometry of the prosthetic component. The cutting sequences calculated could include calculations of machining operations and milling operations.

A CAM software program, namely internal machining software 88, then generates the internal machining data 90 geometries and tool paths for the installation channel 40 and fixing channel 45 and geometry of the fastener seat 50. The internal machining data also specifies the specific tools described above, such as a ball-nose endmill. The software 88 will also calculate the required fastener head clearance required of the installation channel in order to be able to insert the screw through the inclined installation channel and onto the fastener seat. Furthermore, the software will calculate the radius of curvature of the fastener seat, which data is also required in order to clear the screw head to be able to properly access and sit in the fastener seat, while extending through fixing channel 45.

It is understood that while the internal machining (CAM) software 88 is illustrated in FIG. 8 as being in addition to, e.g. a plugin to, the workstation 82, the code in the software 88 could itself be written into the CAD software 83 or the CAM software 86, or provided at any point between user interface and CNC machine 91 to be able to generate internal machining data as described above that can be executed in the CNC machine.

Once the CAM software 88 has generated the required internal machining (CAM) operations of the prosthetic component the generated program is loaded into the CNC machine. The appropriate tools and stock from which the prosthetic component is manufactured is also loaded into the CNC machine and then the controller runs the program based on the generated design data to machine a prosthetic component externally and internally in accordance with the channel and seat geometry described above.

An alternative to manufacture of a system of the invention by CNC machine is to connect a 3-D printer to the controller 82, enabling 3-D printing of the prosthetic component from the CAD data.

The presently described prosthetic system and method and system for making the prosthetic system provides an efficient and reliable as well as accurate process of making a prosthetic system that is a simpler prosthesis for a medical professional to attach to an anchor in a person's body. Automation of the system results in faster manufacturing and precise geometries of the internal channels in the prosthetic component which are configured to maximise ease of installation and ease of manufacturing.

Further embodiments of the system of the invention may include use of the system in non-biological applications, for fixing mechanical components. The system of the invention allows for removal of flanges of material used for bolted connections between components, resulting in reduced material usage, and an increase in part strength. The system of the invention may, for example, be used in the oil and gas industry for pipelines and drilling equipment; in pressure vessels; in shafts, wind turbine blades; and in the aerospace industry for pipe joints and mechanical connections on wings and control surfaces.

Referring again to FIG. 9, which illustrates in block diagram of the system 80, this system may also be used in making the non-biological fastening systems described above. The system 80 includes a controller 82 having a user interface in the form of a scanner, touch screen, keyboard or the like, into which a user can input fastener operating parameters 84.

Examples of fastener operating parameters which can be provided to the controller 82 include scanned three-dimensional models of turbine blade or pipeline. The controller may be in the form of a workstation and includes a three-dimensional CAD (Computer Aided Design) software 83 that will generate a component design to be cut and machined. Other parameters that are provided into the controller include the interface geometry of the anchor to which the component is to be attached. The controller further allows a user to select the location on the surface of the model of the component for the entry point of an installation channel. The user may also use additional tools in the CAD software to modify the model of the component manually, as desired.

The workstation 82 will then calculate and generate design data relating to the external geometry of the component. The designed data calculated will include relative location of the entry points for the installation axis and fixing axis, and the anchor interface profile that complements the anchor to which the component will be attached.

Once the design data has been calculated it is exported to a CAM (Computer Aided Manufacturing) 86 program to generate external machining data 87 for the CNC (Computer Numerical Control) machine 91. The CAM 86 generated external machining data will include tool cutting sequences to create the external geometry of the component. The cutting sequences calculated could include calculations of machining operations and milling operations.

A CAM software program, namely internal machining software 88, then generates the internal machining data 90 geometries and tool paths for the installation channel and fixing channel and geometry of the fastener seat. The internal machining data also specifies the specific tools described above, such as a ball-nose endmill. The software 88 will also calculate the required fastener head clearance required of the installation channel in order to be able to insert the screw through the inclined installation channel and onto the fastener seat. Furthermore, the software will calculate the radius of curvature of the fastener seat, which data is also required in order to clear the screw head to be able to properly access and sit in the fastener seat, while extending through the fixing channel.

It is understood that while the internal machining (CAM) software 88 is illustrated in FIG. 8 as being in addition to, e.g. a plugin to, the workstation 82, the code in the software 88 could itself be written into the CAD software 83 or the CAM software 86, or provided at any point between user interface and CNC machine 91 to be able to generate internal machining data as described above that can be executed in the CNC machine.

Once the CAM software 88 has generated the required internal machining (CAM) operations of the prosthetic component the generated program is loaded into the CNC machine. The appropriate tools and stock from which the component is manufactured is also loaded into the CNC machine and then the controller runs the program based on the generated design data to machine a prosthetic component externally and internally in accordance with the channel and seat geometry described above.

An alternative to manufacture of a system of the invention by CNC machine is to connect a 3-D printer to the controller 82, enabling 3-D printing of the component from the CAD data.

What is claimed is:

1. A system for fixing a prosthetic component to an anchor implanted in bone of a patient, the system comprising:
a fastener configured for location in the prosthetic component for fastening the prosthetic component to the anchor, the fastener having a spherical or part spherical head, and a shaft with a length; and
a prosthetic component having a fixing channel and an installation channel, the fixing channel opening at one end at an anchor engagement collar, and an opposite end opening internally of the component to meet the installation channel at a fastener seat, the fixing channel being configured to accommodate there-through the shaft of the fastener while the head of the fastener sits on the fastener seat, the fastener seat being profiled to receive, and have substantially the same radius of curvature, as the fastener head, the installation channel opening to an outer surface of the prosthetic component and having an installation axis that is inclined relative to a fixing axis of the fixing channel, the axis of the fixing channel intersecting the installation axis at a point that lies within the radius of the fastener seat, said collar being profiled to interlock with the anchor.

2. The system of claim 1, wherein the prosthetic component is a prosthetic tooth, and the anchor is configured for placement in a jaw bone.

3. The system of claim 1, wherein the prosthetic component is a prosthetic joint or at least a portion of a joint, and the anchor is configured for placement in a femur, tibia, acetabular or any combination thereof.

4. The system of claim 1, wherein the prosthetic component is a prosthetic joint or at least a portion of a joint, and the anchor is configured for placement in phalangeal bone.

5. The system of claim 1, wherein the installation channel has parallel sides.

6. The system of claim 1, wherein the fastener seat is located closer to the installation channel opening at the outer surface than the opening at said collar.

7. The system of claim 1, wherein said fixing channel of said prosthetic component has a length along the fixing axis that is longer than a majority of the length of said shaft of said fastener.

8. A method of manufacturing a prosthetic component configured to engage an anchor in a patient, including:
machining a fixing channel into an anchor interface side of the prosthetic component, wherein the fixing channel terminates internally of the component;
profiling the anchor interface side of the prosthetic component to interlock with the anchor;
machining an installation channel from an outer surface of the prosthetic component that meets the fixing channel internally of the component; and
machining a fastener seat through the installation channel at the juncture of the fixing channel and installation channel, including profiling the fastener seat to have a radius of curvature that is substantially the same as a spherical or part spherical head of a fastener adapted to be accommodated on the fastener seat through the fixing channel, the fixing channel having a fixing axis that intersects an installation axis of the installation channel at a point that lies within the radius of curvature of the fastener seat.

9. The method of claim 8, wherein the prosthetic component is a prosthetic tooth, and the anchor is in a jaw bone of the patient.

10. The method of claim 8, wherein the prosthetic component is a prosthetic joint or at least a portion of a joint, and the anchor is in a femoral head, or acetabular, or both, of the patient.

11. The method of claim 8, wherein the prosthetic component is a prosthetic joint or at least a portion of a joint, and the anchor is in a phalangeal bone of the patient.

12. The method of claim 8, further including:
machining at least a second fixing channel into the anchor interface side of the prosthetic component, wherein the, or each further fixing channel at the anchor interface side defines at least a further collar of the prosthetic component configured to be inserted below a bone line into the anchor, and wherein the, or each further fixing channel terminates internally of the component;
machining at least a further installation channel from an outer surface of the prosthetic component that meets the, or each further fixing channel internally of the component; and
machining at least a further fastener seat through the, or each further installation channel at the juncture of the, or each further fixing channel and the, or each further installation channel, including profiling the, or each further fastener seat to have a radius of curvature that is substantially the same as a spherical or part spherical head of at least a further fastener adapted to be accommodated on the, or each further fastener seat through the, or each further fixing channel; wherein the, or each further fixing channel has a fixing axis that intersects an installation axis of the, or each further installation channel at a point that lies within the radius of curvature of the, or each fastener seat.

13. The method of claim 12, wherein the prosthetic is configured for placement on femoral bone and/or tibial bone.

14. The method of claim 8, wherein said installation channel is machined to have parallel sides.

15. The method of claim 8, wherein the fastener seat is machined to be closer to an intersection of the installation channel and the outer surface than to an intersection of the fixing channel and the anchor interface side.

16. The method of claim 8, wherein the fixing channel at the anchor interface side defines a collar of the prosthetic component configured to be inserted below a bone line into the anchor.

17. The method of claim 8, wherein the fastener has a shaft with a length, the fixing channel of the prosthetic component being machined to have a length along the fixing axis that is longer than a majority of the length of the shaft of the fastener.

18. The method of claim 8, further comprising machining a fastener configured to fasten the prosthesis to the anchor, the fastener being machined to have a threaded portion and a non-threaded portion, the non-threaded portion having a length longer than a length of the threaded portion.

19. The method of claim 8, wherein the fastener seat is machined to have a maximum diameter less than a maximum diameter of said head of said fastener.

20. A method of fixing a prosthetic component to an anchor in a patient, including:
machining a fixing channel into an anchor interface side of the prosthetic component, wherein the fixing channel terminates internally of the component;
profiling the anchor interface side of the prosthetic component to interlock with the anchor;
machining an installation channel from an outer surface of the prosthetic component that meets the fixing channel internally of the component;
machining a fastener seat through the installation channel at the juncture of the fixing channel and installation channel, including profiling the fastener seat to have a radius of curvature that is substantially the same as a spherical or part spherical head of a fastener adapted to be accommodated on the fastener seat through the fixing channel, the fixing channel having a fixing axis that intersects an installation axis of the installation channel at a point that lies within the radius of curvature of the fastener seat, the fastener having a shaft with a length;
inserting the anchor interface side of the prosthetic component into the anchor; and
inserting the fastener through the installation channel into the fixing channel to engage the fastener head with the fastener seat and fix the prosthetic component to the anchor.
